(19) United States
(12) Patent Application Publication
(10) Pub. No.: US 2006/0069318 A1
(43) Pub. Date: Mar. 30, 2006

(54) Method for assessment of the structure-function characteristics of structures in a human or animal body

(75) Inventors: Tony M. Keaveny, Berkeley, CA (US); R. Paul Crawford, Sacramento, CA (US)

Correspondence Address: Doyle B. Johnson;REED SMITH LLP, Suite 2000, Two Embarcadero Center, San Francisco, CA, 94111, US

(73) Assignee: The Regents of the University of California

(21) Appl. No.: 11/241,627

(22) Filed: Sep. 30, 2005

Related U.S. Application Data
(60) Provisional application No. 60/614,605, filed on Sep. 30, 2004.

(51) Int. Cl. G06G 7/48; A61B 5/00; G06G 7/58

(52) U.S. Cl. 600/300; 703/11

(57) ABSTRACT

A method for determining one or more structure-function characteristics of a structure in a human or animal body from an image of the structure includes generating a structural model of a structure based on an image of the structure. A first biomechanical quantity is computed based on the structural model. The structural model is varied to create a variant model. A second biomechanical quantity is computed based on the variant model. The first and second biomechanical quantities are compared, in order to assess a structure-function characteristic of the structure.

This application claims the benefit of U.S. Provisional Application No. 60/614,605, filed Sep. 30, 2004, entitled ASSESSMENT OF BONE STRUCTURAL QUALITY, the disclosure of which is herein incorporated by reference.

This invention was made with Government support from an NIH grant, contract number AR41481. The Government has certain rights to this invention.

### BACKGROUND OF THE INVENTION

1. Field of the Invention

The present invention relates generally to a method for assessing the structure-function characteristics of bones and other structures in a human or animal body.

2. Description of the Related Art

According to the National Osteoporosis Foundation and the National Institute of Health (NIH), osteoporosis is a major public health threat for an estimated 44 million Americans—over 50% of the population over age 50—who have low bone mass. In the U.S. today, 10 million individuals are estimated to already have the disease; and the other 34 million with low bone mass are at increased risk for osteoporosis. As the size of the elderly population grows and people live longer, the costs of treating osteoporosis will continue to rise as the disease affects more people. This is also true for other major medical conditions such as arthritis and cardiovascular disease. Osteoarthritis affects an estimated 20 million Americans. An estimated 57 million Americans have some form of cardiovascular disease, which leads to about one million deaths per year (42% of all deaths in the U.S.). Even so, it is difficult to predict heart attacks. It is desired to have an improved method of predicting who is at highest risk, on a patient-specific basis.

Osteoporosis: The current clinical standard for diagnosis and monitoring of osteoporosis, the dual-energy x-ray absorptiometry—or DXA, pronounced “dexa”—scan, has numerous limitations as a technique for assessing the biomechanical integrity of bone. First, DXA scans are two-dimensional areal projections of three-dimensional volumetric bone mineral density information. Thus, areal measurement of mineral density discards potentially important structural effects of how the mineral is arranged and distributed three-dimensionally within a bone. DXA does not differentiate cortical from trabecular bone and is confounded at the spine by the presence of the posterior elements.

Quantitative Computed Tomography (QCT, a variant of a CAT or CT scan), being three-dimensional, overcomes the limitations associated with the planar nature of DXA scans. The three-dimensional nature of the CT scans makes it possible to differentiate mineral density by region or bone type, e.g., the anterior and posterior regions of the bone or the cortical, endocortical, and trabecular bone. However QCT remains a radiological assay and only provides measures of bone density and geometry. Thus, it can be difficult to interpret changes seen in QCT scans.

The mechanical behavior of any structure is fundamentally governed by its geometry and material properties. However, it is difficult to calculate the overall mechanical response of inhomogeneous structures having a non-uniform geometry and non-uniform spatial distributions of material properties—features characteristic of bone and most organs found in the human body. It is also difficult to understand changes in such responses over time or with treatment due to this complexity of geometry and material property distribution.

Whole bones are comprised of two different types of bone tissue: trabecular bone and cortical bone, each with its own unique material strength characteristics. Accordingly, the strength characteristics of whole bones (such as the vertebral body, proximal femur, and distal radius) depend not only on the average density, mass, and size of the bone, but also on the spatially-varying distribution of bone density within the bone, the three-dimensional shape of the bone, and the relative role of the cortical vs. trabecular bone types. In addition, wholes bones in vivo are loaded in complex and multi-axial manners such that they can fail under variable loading conditions. For osteoporotic hip fractures, for example, fractures tend to occur during falls, which produces much different loading conditions on the bone than during habitual activities such as walking. The strength of the bone is also different for these different loading conditions. For spine fractures, the strength of the bone vertebra can be much different for forward bending activities than it is for non-bending activities.

Cardiovascular: For cardiovascular and related applications, techniques such as digital subtraction angiography (DSA) are used to evaluate many vascular regions throughout the body. CT-based angiography is now being used clinically to replace DSA since it is less invasive. Like the application of QCT for analysis of bone in osteoporosis, CT angioplasty does not directly address any of the biomechanical aspects of the underlying clinical problems and thus does not exploit the full potential of the information in the CT images. Restriction of flow in blood vessels might be sensitive to more subtle alterations in the vessel than is apparent by simple visual analysis of the CT angioplasty images. Thus, a biomechanical analysis of blood vessels based on the CT image would provide additional insight into the clinical problem.

Implant Systems: Various types of implants can be introduced into the body to repair the injured or diseased body part. For example, about 150,000 each hip and knee prostheses are implanted each year in the United States, with as many again world wide. Surgeons must choose the most appropriate implant for patients—a difficult choice given that there are many options of devices to choose from for any given medical indication. Use of patient-specific models having associated information on their structure-function characteristics would provide valuable information in choosing an appropriate implant. Similar issues apply to cardiovascular applications such as with stents, in which the appropriate-sized stent is critical to its success. This sizing may depend on the patient-specific biomechanical structure-function characteristics.

Other applications: Other applications having a need for patient-specific structure-function characteristics include arthritis and vertebral fracture repair. For arthritis, improved diagnosis and assessment of treatment would result from knowledge of the biomechanical behavior of the joint, including such effects as patient-specific details on bone density, size, and structure, since stresses that develop in articular cartilage are thought to depend on the density and structure of the underlying bone at the joint. Fracture fixation of the spine can be achieved by injection of bone cement into the affected vertebral body. Such procedures might be optimized by knowing the structure-function characteristics of the resulting bone-bone cement system since the stiffness of the system depends on the amount and location of the injected bone cement. Fracture fixation using metal or other types of prostheses could also be improved by assessing the structural response of the bone-implant system to such factors as size and shape and material of the prosthesis, as well as the density and structure of the body part. In this way, surgeons can evaluate the suitability of a proposed surgical procedure in advance of an operation, thereby choosing the optimal course of action for an individual patient. Currently, most surgeons depend only on their qualitative experience and have little or no quantitative means for evaluating the various options. Knowledge of the structure-function characteristics of the various types of bone-implant systems would result in improved patient outcomes.

### SUMMARY OF THE INVENTION

A method is provided for determining one or more structure-function characteristics of a structure in a human body or animal from an image of the structure. The method includes receiving an image of a structure in a human body or animal. A structural model of the structure based on the image is generated. A first biomechanical quantity based on the structural model is computed. The structural model is modified to create a variant model. A second biomechanical quantity is computed based on the variant model. The first and second biomechanical quantities are compared. A result of the comparing is stored in a digital medium.

The method may further include determining the one or more structure-function characteristics based on the comparing. The method may further include repeating the method for altered loading conditions and/or repeating the method at a later time period, and determining one or more effects of treatment, aging and/or disease on one or more structure-function characteristics of the structure.

The structure may include a musculoskeletal tissue or organ such as bone, or a cardiovascular tissue or organ such as a heart or blood vessel, which may or may not have an attached implant. The variant model may include a homogenized model, wherein the method includes assigning an average density to one or more elements of the structural model.

The variant model may include a reference model, wherein the method includes assigning a reference density to the structural model. The variant model may include a sub-structure model, wherein the method includes removing a portion of bone from the model and determining a structure-function characteristic of the remaining bone. The portion of bone that is removed may include peripheral bone or internal bone.

The variant model may also include an axial model or a bend model. The variant model may include a combination of two of more of a homogenized model, a sub-structure model, an axial model, a bend model, and a reference model.

The variant model may include a variation of the structural model wherein a boundary condition is modified. The boundary condition may include force, pressure, deformation, fluid field, energy, and/or stress.

The method may further include scanning the structure to create the image of the structure. The scanning may include computed tomography (CT) or magnetic resonance image (MRI) scanning, and the structural model may include a finite element model of the structure. The method may further include receiving a second image of the structure acquired at a different time period than when the first image was acquired. A second structural model of the structure may be generated based on the second image. A third biomechanical quantity may be computed based on the second structural model. The second structural model may be modified to create a second variant model. A fourth biomechanical quantity may be computed based on the second variant model, and wherein the comparing may further include comparing the third and fourth biomechanical quantities. The comparing may also include comparing the result of the comparing of the first and second biomechanical quantities with a result of the comparing of the third and fourth biomechanical quantities.

The method may further include receiving a second image of a different structure. A second structural model may be generated based on the second image. A third biomechanical quantity may be computed based on the second structural model. The second structural model may be varied to create a second variant model. A fourth biomechanical quantity may be computed based on the second variant model, and wherein the comparing may include comparing the third and fourth biomechanical quantities. The comparing may also include comparing a result of the comparing of the first and second biomechanical quantities with a result of the comparing of the third and fourth biomechanical quantities.

In another embodiment, the present invention includes a method for determining the efficacy of a treatment on a structure in a human or animal body, the method may include receiving an image of a structure in a body at a first time period, generating a structural model of the structure based on the image, computing a first biomechanical quantity based on the structural model, modifying the structural model to create a variant model, computing a second biomechanical quantity based on the variant model, comparing the first and second biomechanical quantities, and determining one or more structure-function characteristics based on comparing the first and second biomechanical quantities, receiving a second image of the structure acquired at a second time period, generating a second structural model of the structure based on the second image, computing a third biomechanical quantity based on the second structural model, modifying the second structural model to create a second variant model, computing a fourth biomechanical quantity based on the second variant model, comparing the third and fourth biomechanical quantities, and comparing a result of the comparing of the first and second biomechanical quantities with a result of the comparing of the third and fourth biomechanical quantities, in order to determine the efficacy of a treatment on the structure.

The technique can be applied to humans or animals. It could also be applied to scans taken in vivo or ex vivo. While clinical diagnostics can only be forthcoming from in vivo scans on humans, insight into treatments, aging, and disease can be obtained by applying this invention to animal and cadaver studies.

One or more processor readable storage devices are also provided having processor readable code embodied thereon. The processor readable code programs one or more processors to perform any of the aforementioned methods.

### BRIEF DESCRIPTION OF THE DRAWINGS

The present invention will be readily understood by the following detailed description in conjunction with the accompanying drawings, wherein like reference numerals designate like structural elements, and in which:

FIG. 1 illustrates outcome variable in finite element parameters studies on vertebral strength used to determine bone strength and/or bone quality.

FIG. 2 includes a plot of trabecular compartment strength versus whole vertebral strength.

FIG. 3A includes a plot of homogeneous bone strength versus standard bone strength.

FIG. 3B includes a plot of standard to homgeneous bone strength ratio versus standard bone strength.

FIG. 4 illustrates a process in accordance with a preferred embodiment.

FIGS. 5A-5D illustrate variations in the microstructure of trabecular bone.

FIG. 6 illustrates a cross-section of a trabecular bone showing variations in the bone mineral density within the individual trabeculae.

### DETAILED DESCRIPTION OF THE INVENTION

The following description is provided to enable any person skilled in the art to make and use the invention and sets forth the best modes contemplated by the inventor for carrying out the invention. Various modifications, however, will remain readily apparent to those skilled in the art. Any and all such modifications, equivalents and alternatives are intended to fall within the spirit and scope of the present invention.

Because of the limitations with using DXA-measures of bone density to both predict those who are highest risk of fracture and to assess the efficacy of treatments, researchers are seeking methods to quantify what aspects of “bone quality” are changing with age, disease, and treatment. Accordingly, a method of quantifying aspects of whole bone quality is provided herein. A benefit is improved diagnosis of osteoporosis, monitoring of treatment, and assessment of new treatments which can each benefit those afflicted with osteoporosis.

In this context, it is useful to define bone quality in terms of “structure-function characteristics”. By “structure-function characteristics”, it is meant here any relation between measures of the whole bone structure that is imaged and any aspect of the biomechanical behavior of the whole bone structure. The term “structure” in this invention can also be interpreted to comprise any portion of a human or animal body, for example an organ such as a bone or the heart or the intervertebral disc or a human joint such as the hip or knee joint, or a portion of any such organ. Structure can also refer to a tissue such as trabecular bone, cartilage, intervertebral disc material, ligament, tendon, blood vessel, or any other tissue in the body including fluidic components such as bone marrow, blood, or any other bodily fluids. It is understood therefore that the term “structure” as used in this invention refers to any part of the body, at any physical scale. A structure could also include an implant attached to some part of the body, for example a bone-implant system comprising of an artificial hip implant implanted in the proximal femur, or a cardiovascular stent implanted within a blood vessel. Any quantitative measure that characterizes the geometry, morphology, mass, or density of the structure can be used to quantify structure in the characterization of the structure-function relations. For example, structure could be characterized by such measures as body-weight, height, bone density, bone density distribution (characterized by the standard deviation of a spatial distribution of bone density values within the bone), hip-axis length, blood vessel diameter, variation of thickness along a length of a blood vessel (as characterized by a standard deviation of thickness values along the length of the vessel), cartilage thickness, muscle orientation and length, muscle cross-sectional area, trabecular number, trabecular connectivity, or fluid viscosity.

The term “function” refers to any biomechanical characteristic of the structure, calculated by biomechanical analysis of the image. This includes but is not limited to such mechanical parameters as strength, stiffness, fatigue resistance, fracture toughness, toughness, deformation, risk of fracture, fluid shear stress, and pressure. In biomechanical problems in which temperature and heat transfer are involved, such parameters as temperature gradient, heat flux, and thermal expansion stresses would also be understood to be quantitative measures of “function”.

The “structure-function characteristics” of this invention are any mathematical relations of any sort between quantitative measures of structure and the quantitative measures of function. For example, the density-strength relation—a plot between strength values and density values for a number of bones—is a well-known structure-function relation for bone. The ratio of the strength to the density for a single bone or for a single piece of bone tissue represents another manifestation of the structure-function characteristics. The relation between blood vessel diameter and shear stress on the walls of the blood vessel represents another example of the structure-function characteristics.

In addition, “structure-function characteristics” in this invention refers to any mathematical relation between any biomechanical parameters calculated using variants of the models of the image of the structure, or any combination of such, with either: a) any other biomechanical parameters calculated using variants of the models of the image of the structure; or b) any of the quantitative measures of structure or quantitative measures of function such as those described above. For example, the ratio of trabecular strength after removing 2 mm of peripheral bone from a model of the human vertebral body to the strength of the (intact) human vertebral body, would be considered a structure-function characteristic.

With many new drugs in the development pipeline, an assessment tool in accordance with a preferred embodiment provides tremendous advantage. The approaches described herein provide such tools. In one embodiment, information can be used clinically to help optimize treatments for individual patients. Currently, there are a number of osteoporosis drug treatments on the market, some having much different biological actions (for example, alendronate reduces bone resorption whereas PTH invokes new bone formation). As more treatments become available, patients will have more choices as to which drug to take. The information provided by the methods disclosed herein can provide clinicians with the tools to tailor each drug treatment to a specific patient.

A variety of methods are provided herein for quantifying biomechanical structure-function characteristics for bone strength and using them as part of diagnosis, monitoring, and assessment of diseases such as osteoporosis and their treatment. This is done by performing controlled parameter studies on a patient's bone to quantify its strength or other biomechanical properties for a variety of altered conditions. In a preferred embodiment, the bone or bone data is altered in a controlled fashion for an individual patient to produce data that is then used on a patient-specific basis for the diagnosis of osteoporosis or the assessment of therapeutic treatments over time. A comparison of a bone's structure-function characteristics against a database of those derived from normal, healthy bones or against that behavior of the same bone at an earlier time point can form a more detailed and informative basis for assessment of disease and therapy effects compared to what is provided by use of single metrics of bone mass, density, or even strength. Changes in the structure-function characteristics of the bone can be quantified at a particular time point (cross-sectionally) and tracked over time (longitudinally), the latter being particularly insightful in understanding the biomechanical mechanisms associated with therapeutic treatments of bone strength or in the progression of a disease or with aging.

A method in accordance with a preferred embodiment has more general applicability than to just assessing bone strength and osteoporosis. Such method is applicable to applications in which bone strength is relevant, and to other tissues and organs in which mechanical behavior of a tissue or organ is important clinically. In cardiovascular disease, for example, analysis of structure-function relations for blood vessels may lead to improved diagnosis of heart disease by quantifying a risk of clotting by the presence of or by the disruption of the mineralized plaque, or of rupturing of a blood vessel. In arthritis, biomechanical analysis of cartilage provides diagnostics that can better predict the response to surgery or drug treatment. It also provides improved tracking of the disease in patients and better monitoring of any treatments. The biomechanical response of the body to the implantation of an artificial joint or biomaterial or tissue-engineered construct or any substance may be monitored in greater detail, such that the suitability of the implanted item to a specific patient is improved.

A preferred technique utilizes non-invasive, patient-specific techniques and modeling to assess bone strength. A controlled set of parameter studies is preferably used on such models in order to provide additional data that is then used in a patient-specific manner for diagnosis or assessment of treatment or tracking of a disease or any change in the tissue or organ. The general concept can be applied to both tissues, such as trabecular bone, and organs, such as a proximal femur or a vertebral body. It can also be applied to a biomechanical system in which a patient-specific image is used to assess mechanical behavior in a non-invasive manner.

Specifically, a method in which parameter studies are run on patient-specific finite element models of biological structures or materials such as bones, cartilage, tendon or other musculoskeletal tissues, or blood vessels in order to characterize the biomechanical structure-function characteristics is disclosed. Any non-invasive technique can be used to assess the mechanical behavior, such as any combination of 3D or 2D imaging to acquire the patient-specific image, such as but not limited to MRI, CT, ultrasound, QCT, micro-CT, micro-MRI, and finite element modeling, beam theory or other analytical modeling that uses principles of continuum mechanics or mechanics of materials or fluid mechanics or heat transfer or mass transport or thermodynamics to produce the mechanical response.

As an example, for analysis of a whole bone, a preferred technique provides quantitative measures of how various elements of the whole bone contribute to its strength. The very strong nature of the correlations that can exist in these relations (see FIG. 2) provides a technique to identify bones that do not fall within a typical range of structure-function behavior for normal bone even though bone strength appears normal. This approach provides a more sensitive measure of the biomechanical status of the bone than use of a single strength metric (such as compressive strength) and can provide additional and unique insight into evaluation of the biomechanical effects of aging, disease, and drug therapy or other treatments, since these may alter the natural structure-function characteristics for whole bones. This analysis can be done at one point in time, or, be repeated over time.

The patient-specific nature of the finite element modeling or any mechanical analysis used for this solution arises from the generation of such models from non-invasive imaging procedures applied to the tissue or organ, such as but not limited to computed tomography (CT), magnetic resonance imaging (MRI), dual energy absorptiometry (DXA), positron emission tomography (PET), micro-CT, peripheral CT, quantitative CT (QCT), or any type of scan or combination of scans that produces a 2D or 3D image of the patient's tissue or organ. Once a computer model of the tissue or organ is generated, a relevant mechanical property is computed or calculated, e.g., typically stiffness or strength. The scan can also be analyzed to quantify various aspects of the density and/or geometry or morphology of the tissue or organ.

The computer model is then altered in one or more of various controlled ways and resulting mechanical property values are computed. Various structure-function characteristics are quantified by plotting the mechanical properties against each other and/or the density and geometry properties. Certain mathematical functions of these, for example a ratio of strength to density, or a ratio of one derived mechanical property to another or to strength or to density, also represent elements of the structure-function characteristics. The use of a structure-function characteristic as an assay of the tissue/organ status is advantageous in addition to the primary mechanical property (e.g., strength or stiffness). In this way, controlled parameter studies on the original model are used to provide new data from the medical image on a patient-specific basis that can be used to enhance the diagnosis of a disease or the assessment of a treatment or an altered or aged state.

For example, in assessing the effects of osteoporosis drug treatments on trabecular bone microstructure, one could perform finite element modeling using high-resolution micro-CT scans of a patient's wrist. By performing controlled parameter studies on that patient's bone using the computer model, it is possible to extract the independent role of any changes in the distribution of mineralization from those in geometric microstructure. This can provide additional insight into the biomechanical action of the drug treatment. Similarly, one could assess the mechanical integrity of a blood vessel under different degrees of mineralization using information taken from clinical CT scans of the patient's heart.

A detailed example of the procedure as applied to analysis of a vertebral body in the assessment of a drug treatment is described next, realizing that the invention is not limited to this specific application.

A patient is scanned with QCT, which produces a digital image of their vertebral body. The QCT image of the vertebra or interest, for example, an L1 or T9 vertebral body, is then converted into a finite element model, following such procedures as described in Crawford et al. (Crawford et al.: “Finite element models predict in vitro vertebral body compressive strength better than quantitative computed tomography.” Bone, 33:744-750, 2003) or equivalent. Briefly, geometry information in the scan is used to create a finite element mesh of the bone. Mineral density information in the scan is used to generate material properties for each finite element in the model on an element-by-element basis. Elastic and strength properties of the bone tissue are assigned to each bone element in the model (see, for example, Crawford Trans Orthopaedic Research Society 2004; or Faulkner et al.: “Effect of bone distribution on vertebral strength: assessment with patient-specific nonlinear finite element analysis.” Radiology 179:669-674, 1991).). Finite element model boundary conditions associated with applied loads of interest are applied to the model, and the model is solved by a computer to determine strength or other structural properties of interest, e.g., stiffness or deformation.

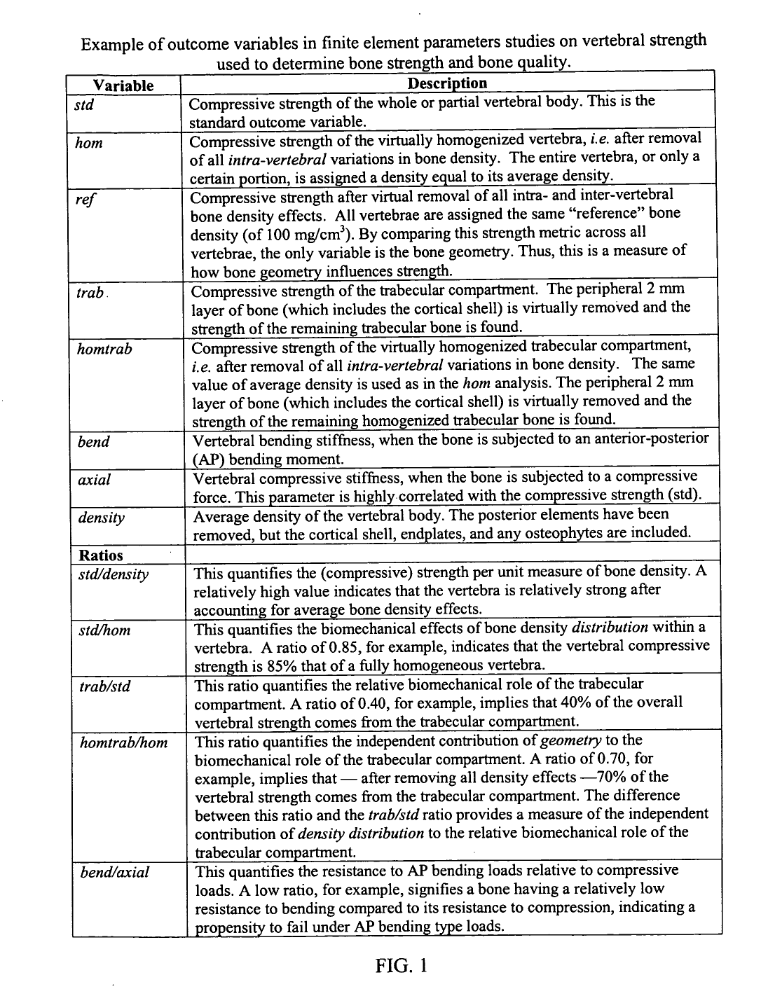

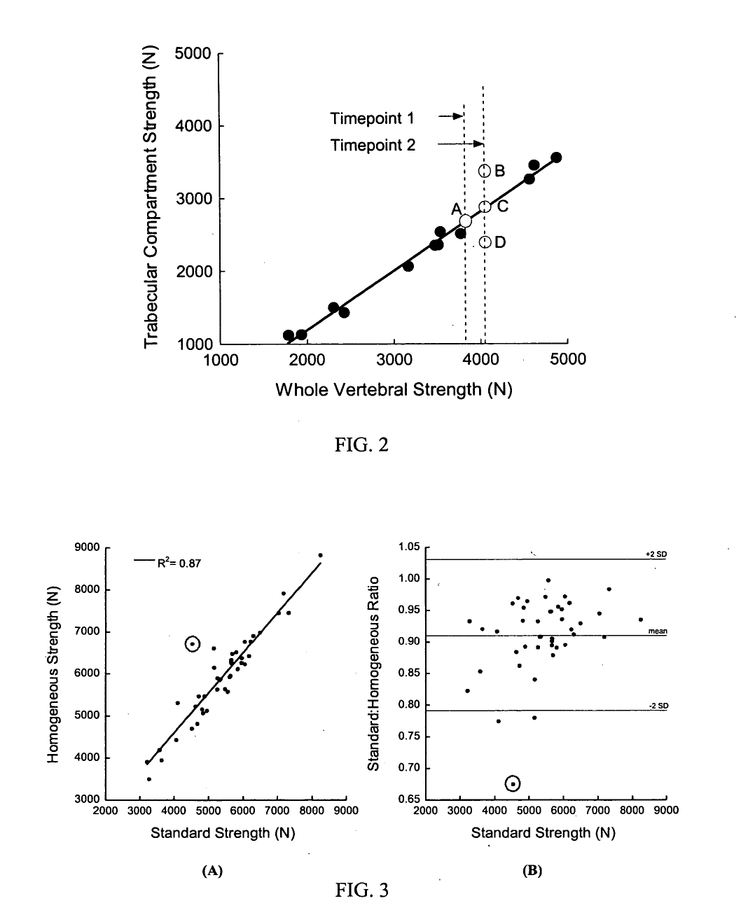

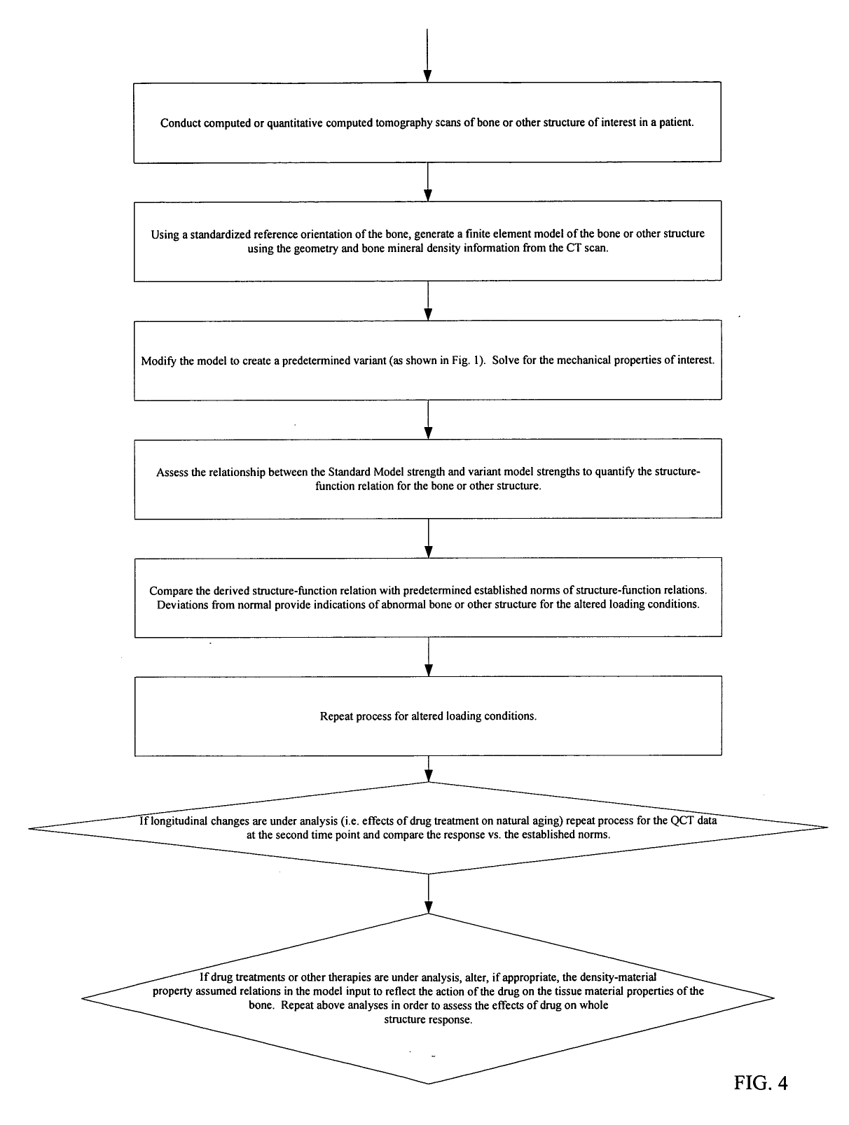

Next, a number of parameter studies are done on the original model to vary the model and compute the resulting values of strength (or relevant outcome parameter in terms of whole bone mechanical behavior). Such parameter studies could include those shown in FIG. 1, in which seven separate finite element analyses are run to provide a total of 13 outcome parameters, five of which are derived ratios.

In one such analysis, the outer 2 mm of bone is removed and the strength is computed of the resulting trabecular compartment. This quantifies the structural role of the trabecular compartment (variable trab in FIG. 1). FIG. 2 shows an example of a structure-function relation for the human vertebral body in a healthy group of women in which the strength values are computed by patient-specific FEM, and strength is shown after erosion of the 2 mm of peripheral bone for a group of normal human vertebrae. The data is a sample of 13 normal healthy human vertebral bodies, showing a plot of the strength of the trabecular bone compartment vs. the strength of the whole vertebral body. Three hypothetical cases are shown for changes in behavior for an individual that occur over time, due to aging, disease, or treatment. The high R² value of this relation (R²=0.99) indicates that relatively small deviations from this relation are indicative of behavior outside normal structure-function behavior. Such deviations could be interpreted as changes in whole bone “quality” and may be useful clinically.

FIG. 2 also shows three possible hypothetical cases of changes in structure-function characteristics that may occur due to aging, disease, or treatment. Analysis of changes in this way can be used to understand the biomechanical mechanisms of the changes, and can provide insight into, and indeed quantify, changes in quality of the whole bone. Starting at point A as the initial time point, the individual could move to three points: for point C, since it lies on the regression line of “normal” structure-function behavior, there is no change in quality. For point D, which falls noticeably below the regression line, the strength of the trabecular compartment is relatively low, signifying a change in the structure-function behavior. For point B, the strength of the trabecular compartment is relatively high, again signifying a change in the structure-function behavior. In each case, the strength of the whole vertebral body increases the same amount, signified by the same final values of vertebral strength for each case. Case B, for example, if found in a drug study, would show that a drug treatment achieves its overall strength-enhancement effects by preferentially increasing the strength of the trabecular compartment. This information helps explain the biomechanical action of the drug treatment.

In a related variation of the model, a sub-structure model is created in which some portion of the bone is removed, not necessarily the peripheral bone. For example, in a model of the vertebral body, an inner core of trabecular bone could be removed, and the diameter of this core could be sequentially increased, removing various amounts of bone internally from the whole bone.

In another variation of the model, some or all of the bone material within the model is assigned the average density of that vertebral body. In this way, the bone is homogenized and some or all of the intra-vertebral variations in density are eliminated. As a result, the material properties of the bone material with the vertebral body are also homogenized and the same values are used throughout the vertebral body. The strength of this new model (hom in FIG. 1) is computed. The ratio between this strength value and the strength computed previously for the intact vertebral body (std in FIG. 1), a quantitative aspect of the structure-function characteristics, provides a measure of the importance of the variation of density within the vertebra. Typically for a vertebral body, the std/hom ratio is less than one (FIG. 3). The homogenization can be done in different degrees in order to remove some or all of the variations in density.

As the bone becomes more homogeneous, this ratio approaches a value of one and the bone becomes more optimal from a structural perspective. Thus, values close to one indicate a structurally efficient bone; lower values denote a bone that is not so well optimized. A drug treatment may alter this ratio, signifying an alteration in the overall structure-function characteristics of the bone with treatment. If the ratio increased with treatment, for example, that would suggest that the treatment increases the structural efficiency of the bone, i.e. maximize strength for a given amount of bone mass. It is theoretically possible that a bone could lose strength but increase in its structural efficiency. Thus, the std/hom ratio provides additional information than just the measure of strength.

This information can also be used to choose the most appropriate treatment for a patient. For example, if a value of 0.90 is found for this ratio for a given patient, that indicates that a drug treatment does not have to alter the spatial distribution of bone for this patient since they possess a structurally efficient bone (but they may have low bone strength nonetheless). By contrast, a patient with a ratio of 0.70 would have a poor structural efficiency. Treatment for this patient would tend to homogenize the spatial distribution of bone density in order to optimize the strength of the given bone mass. In practice then, a physician would make measurements of these ratios in patients, and depending on how close that ratio is to a value of one (the data points shown in FIG. 3B could be used to determine what is “normal”), they would recommend the type of drug treatment for the patient. The use of these new metrics is also advantageous.

In the graph of FIG. 3A, a plot of the homogenized strength vs. the standard strength is shown. This is another aspect of the structure-function characteristics. The circled point exhibits behavior outside the average behavior of the other specimens because it falls off the main regression line. In FIG. 3B, the plot shows, for the same data, the ratio of standard to homogenized strength plotted against the standard strength. This is another aspect of the structure-function characteristics. Values of this ratio that approach a value of one represent vertebrae with structurally efficient behavior; low values represent vertebrae with inferior density distribution. The same circled point is much more evident here as an outlier. The horizontal lines show the mean and ±2 SD values of the standard to homogenized strength ratio, identifying two other outlier points that were not so evident on the left plot. Because the ratio of the standard to homogeneous strength for the highlighted specimen is so low, although it has only a slightly below-average value of strength, it has poor quality because its density distribution is not efficient at providing strength.

In another variation of the model, bone material within the model is assigned a reference value of a predetermined density (say, 100 mg/cm³, but the actual value is arbitrary). In this way, intra-vertebral and inter-vertebral variations in density are eliminated, so that vertebrae analyzed have the same density (in the homogenized models, each vertebra is assigned its own unique value of average density). As a result, there is no substantial difference in material properties within or across vertebrae. When the resulting “reference” strength of these models (ref in FIG. 1) is compared across bones, a relevant difference is due to the geometry of the bones. Thus, this analysis isolates the independent effects of bone geometry on strength. Comparison of the “reference” strength against the “standard” strength for different vertebrae is another example of quantification of the structure-function characteristics. A drug treatment may alter the ref strength measure, signifying an alteration in the overall strength due only to changes in geometry. This parameter represents another aspect of the structure-function characteristics of the bone. This measure is particularly useful in complex geometrical structures such as bones because it can be difficult to know a priori what aspect of the geometry is most relevant from a strength perspective. The ref strength measure integrates the overall effects of geometry and thus it is not necessary to specify a priori any particular geometric feature (such as height, width, etc).

In practice, such measures may be helpful in diagnosis or assessment of treatments. For example, it is very difficult in diagnosis of osteoporosis and fracture risk for the hip to choose any single geometric feature for assessment of how the geometry of the bone is changing with aging or disease. This is due to the complex geometry of the proximal femur. The ref strength measure overcomes this limitation. Thus another embodiment involves the use of the ref strength measure to quantify net geometric biomechanical effects of changes that might occur in a bone or other organ or tissue. Comparison of the ref strength at different time points represents another example of the structure-function characteristics.

In another variation of the model, the loading conditions on the bone can be altered. For example, a bending moment is applied to the bone in one case, and compared to a uniform compressive loading in another case. The ratio of these (bend/axial in FIG. 1) provides a relative measure of the resistance to one loading mode over the other. The bend/axial ratio, for example, may increase with a drug treatment, signifying that the bone becomes more resistance to bending type loads with treatment. Again, this provides more information than just stating that the bone increases its strength with treatment. Also, it is theoretically possible for bone strength to decrease with a treatment (or with aging or disease) but for the bend/axial ratio to increase. Thus, this ratio provides additional information beyond consideration of just the individual measure of strength. In practice, patients with normal values of compressive strength but with low values of bend/axial may be considered at higher risk of fracture. Thus, use of this ratio in the diagnosis and assessment of treatment in accordance with a preferred embodiment provides significant advantage.

In another aspect, the relation used to map bone density to bone material properties—an input feature used in converting QCT or any other medical scans into biomechanical finite element models or other biomechanical models (including analytical models)—can be altered to reflect the action of a drug or other treatment. Other research may provide information on changes in these relations for the bone tissue, for example. In this way, the effects of how treatments can affect the material properties of the bone tissue can be integrated into a structural analysis of the whole bone and its effects on whole bone strength can be quantified. Also, by comparing the response of the bone with vs. without the altered density-mechanical property relation for the material properties, it is possible to isolate any changes in strength due specifically to this alteration. This enables quantification of the effects of alterations of the structure-function properties of the bone material on the whole strength of mechanical behavior of the vertebral body.

In these analyses, the percent load capacity associated with the model changes is quantified by comparison against the original model that was generated (the Standard Model or its equivalent in FIG. 1). Other methods of expressing the data could be used, for example, absolute measures of load capacity in the altered model, or absolute difference vs. the Standard Model or any other model. The data point for the bone under analysis is then compared against the population response to determine if the test bone falls within the normal range of structure-function behavior. The data point can also be compared over time against previous measures for the same patient. The data point could also be compared against a bone in another part of the body, for example, comparing the right and left femurs of an individual, one of which might be differently altered by a treatment. The entire process is preferably automated via a computer program.

The flowchart of FIG. 4 illustrates processes that may be performed in various combinations in accordance with preferred and alternative embodiments:

1. Obtain computed or quantitative-computed tomography digital scans of a bone or other structure of interest in a patient.

2. Using a standardized reference orientation of the bone or other structure, generate a finite element model of the bone or other structure using the geometry and bone mineral density information from the CT scan.

3. Modify the model to create the aforementioned variants. Solve for the biomechanical properties of interest.

4. Assess the relationship between the variant model strengths with each other or with the Standard model strength or between any of these measures and any quantitative measures of geometry, density, or morphology in order to quantify the structure-function characteristics for the bone or other structure.

5. Compare to established norms of structure-function relations. Deviations from normal provide indications of abnormal bone for the specific loading configuration of interest.

6. Repeat process for altered loading conditions.

7. If longitudinal changes are under analysis (for example, effects of drug treatment or natural aging) repeat the above process for the CT data at the second time point (i.e. after treatment or time) and compare the response vs. the established norms.

8. If site-effect changes are under analysis (for example, effects of drug treatment on different parts of the body) repeat the above process for the CT data of the second body part and compare the responses with each other and vs. the established norms.

9. If drug treatments or other therapies are under analysis, alter, if appropriate, the density-material property assumed relations in the model input to reflect the action of the drug on the tissue material properties of the bone. Repeat above analyses in order to assess the effects of the drug on whole bone response.

In clinical practice, these processes are preferably highly automated computationally, and performed on a computer running software programmed according to the aforementioned embodiments. The outputs of any and/or all steps, including a final output, may be stored in a digital medium.

In addition, it is possible to add specificity to the assessment of age, disease, or treatment effects by tracking changes in biomechanical function at a specific point in the bone that can be mapped between different timepoints. For example, changes in a localized region of bone from one timepoint to the next will make it possible to more specifically isolate where and how structural changes are being achieved. If the geometry of a finite element at a second timepoint is used with the corresponding finite element material properties at an initial timepoint, the geometric effects of a treatment are more specifically isolated. Similarly, if the material properties of a finite element at a second timepoint are used with the corresponding finite element geometry at an initial timepoint, the changes in the material properties and their distribution effects are more specifically isolated.

The concepts here have been described for application to bone, and to osteoporosis in particular. The general method of performing such controlled parameter studies on a patient-specific basis and comparing against structure-function characteristics for population norms or against itself over time can be applied to an application in which biomechanical computer models are generated from patient scans (e.g., QCT, pQCT, CT, micro-CT, MRI, micro-MRI, US, DXA, PET, X-ray, including any combination thereof). A form of biomechanical analysis can be used to produce non-invasive measures of strength or other biomechanical characteristics of interest (e.g., stiffness, stress, deformation, energy absorption, fatigue characteristics, toughness, fracture toughness, crack propagation characteristics, fluid stress, pressure). Such analysis techniques are not limited to the finite element method, and could include a form of analytical modeling, beam theory or composite beam theory or another form of beam theory, fracture mechanics, composite material analysis, or a branch of continuum mechanics including solid and fluid mechanics, heat and mass transfer analysis, dynamic analysis, or another branch of mechanical analysis, or another branch of numerical analysis including the finite difference method. For example, analysis of bone-implant systems could vary the bone-implant interface conditions, or the material properties of the bone and/or implant. Models in blood flow analysis could vary the elastic properties of the blood vessel, change the flow conditions, alter the geometry of blood vessels, alter the geometry of junctions between blood vessels, or alter the geometry of plaque or other blockages, including adding such obstructions to blood flow. In each case, the appropriate medical image and engineering theory would be used.

The present method could also be applied to simulate hypothetical changes in the future in order to assess risk or suitability of possible future conditions. This would be useful in surgical planning, deciding on what drug treatment to use, and for consideration of prophylactic treatment. For example, in vertebroplasty and similar medical procedures, bone cement is introduced to repair fractures and reinforce a bone. By varying an image-based model of a patient's bone to include different amounts or locations of introduced bone cement, and measuring the resulting changes in whole bone strength or deformation under assumed loading conditions and plotting those characteristics against each other or the relative volume of the introduced bone cement, the structure-function characteristics of the resulting bone-implant system could be quantified. This information could be used to decide on an optimal treatment for the patient in terms of choosing the appropriate amount of bone cement material to be introduced or to specify a location within the bone for introduction of such bone cement.

In another bone-implant example, a model of the image of a patient's bone before implantation could be altered to include the implant. Biomechanical parameters such as stress in the bone could be compared before and after the implantation. The material properties, geometry, position, or size of a to-be implanted prosthesis could be varied. An implant would then be chosen according to some criterion, for example, bone that minimizes reduction in bone stress for a given set of loading conditions. This would help the surgeon choose the correct implant configuration to use for a specific patient.

As another specific example, the following describes the present technique as applied to analysis of trabecular bone, using analysis of models generated from micro-CT scans of bone or micro-MRI scans of bone. Newitt et al. (Osteoporosis International 13:278-287, 2002) described how finite element models derived from patient high-resolution MRI scans of the distal radius (wrist) were used to non-invasively estimate the anisotropic elastic properties of the trabecular bone. Using such an analysis technique or any other means of producing a finite element model or other type of structural model from the images, such models can be altered in a controlled fashion in order to determine mechanisms of action of treatments by quantifying the structure-function characteristicss. In one variation, the standard model would be generated according to Newitt based on a micro-MRI scan of the patient's wrist, in which a small specimen of trabecular bone from the wrist would be isolated and the standard strength (or equivalent) analysis would be run. This model would then be varied by removing the outer voxels on each individual trabecula in order to determine the structural role of the outer bone tissue. The relation between the strength of the original model vs. the strength of the model with the removed bone would quantify a structure-function characteristic for the bone. In another variation, one or more individual trabeculae could be removed or added. In another variation, a layer of bone could be added to simulate possible effects of a particular type of drug treatment; or bone could be added according to some type of pre-determined criterion that is known from other studies on the action of various treatments. In another variation, small cavities could be added along trabecular surfaces to probe the effects of resorption spaces on overall mechanical properties. In another variation, such resorption spaces could be filled in and the resulting mechanical properties computed. It is thought that creation of new resorption spaces, removal of individual trabeculae, or filling in of existing resorption spaces are all possible mechanisms by which disease and treatment can affect the mechanical properties of trabecular bone. In another variation, bone material could be removed until a certain number of individual trabeculae are lost, or, a certain mass of bone could be removed according to some pre-determined criterion. In all cases, the mechanical properties of the bone before and after changes are computed and the structure-function characteristics are quantified. This could be done as a diagnostic tool or to assess treatments or to investigate the action of new or poorly-understood treatments.

These controlled parameter studies could provide a technique to tailor a treatment to a specific patient. It is possible, for example, that a patient's bone may be more prone to the loss of individual trabecular struts, which may identify them as more suitable for one type of treatment over another. Another type of patient may be less responsive to filling in of micro-cavities associated with anti-resorptive or similar treatments, and thus may be more suitable for a different type of treatment.

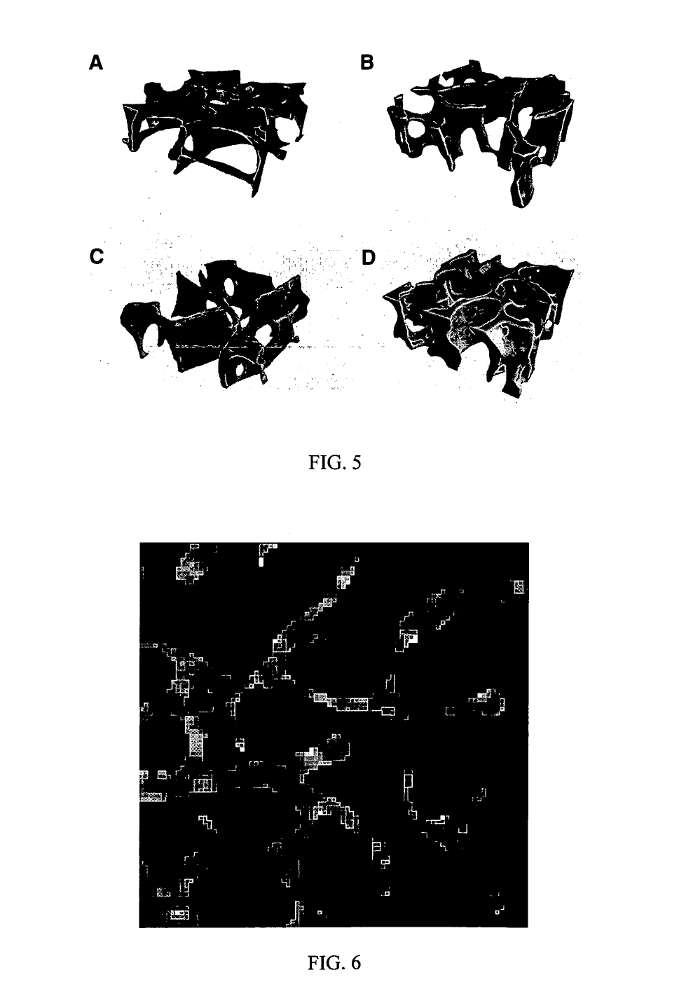

Micro-CT and finite element modeling can also be used clinically to assess the strength and elastic properties of trabecular bone using the new generation of micro-CT and peripheral-QCT scanners for clinical usage (e.g. Extreme CT from Scanco, Inc.). Using such an analysis technique, one can generate models of the trabecular bone microstructure, on a patient-specific basis (see FIG. 5). These images can then be converted into finite element or other models to compute strength or stiffness of the overall bone specimens. Typically, this would be done at the distal radius, but sites such as the calcaneus and tibia are also possible. One such application has been described by Stauber et al. (“A finite element beam-model for efficient simulation of large-scale porous structures.” Comput Methods Biomech Biomed Engin. 7:9-16, 2004). This technique can be applied to patients non-invasively, or to biopsies obtained from patients, or, to cadaver material in basic science studies, or, to animals in pre-clinical trial research studies.

A typical cross-section of a 3D micro-CT based finite element model having variations in bone mineral density is shown in FIG. 6. Finite element analyses can be done on such models to estimate their strength or stiffness or other mechanical characteristics non-invasively. According to another embodiment, one would then perform controlled parameter studies to quantify the structure-function characteristics.

According to another embodiment, after generating a standard model from the micro-CT scan, one would perform a set of controlled parameter studies on this model. The same variations could be made as described above for the micro-MRI models. Unlike MRI scans, CT scans can provide quantitative data on the degree of mineralization of bone tissue. Thus, another controlled variation could be done in which the bone mineral at each voxel could be averaged, and then used in some or all elements, similar to the hom analysis described earlier on the clinical QCT models of the whole bone. By comparing analyses with and without the spatial variations in mineralization, one can extract out the mechanical response to changes in the spatial distribution of mineralization. In a further variation, similar to the ref case described above for the vertebral body, trabecular bone in patients is assigned a fixed value of bone density, thus isolating the effects of micro-structural geometry.

FIG. 6 illustrates a typical cross-section of a 5 mm cube specimen of trabecular bone imaged at about 70 micron spatial resolution using micro-CT. White regions represent more dense bone which is more highly mineralized; the gray regions represent regions of lower density which are less mineralized. The pure black regions represent bone marrow. To assess the independent effects of the spatial variations in mineralization within the specimen, the specimen can be homogenized—assigned the same value of density in some or all elements—and the strength of the homogenized specimen can be compared against the strength of the same specimen without homogenization.

Embodiments techniques described herein provide techniques to more comprehensively, sensitively, and/or specifically identify bone that is diseased or structurally abnormal. A process is further provided to assess the biomechanical pathways by which aging, disease, and various treatments, past and future, affect whole bone strength. Embodiments can be applied not only to whole bones, but also to bone tissue and any other tissues or organs for which biomechanical behavior is clinically relevant.

For cardiovascular application, a model of a patient's blood vessel could be created from various forms of MRI or CT images, including their combination. Such a model would include a patient-specific description of the geometry of the vessels under examination. The presence of plaque could also be evident. Finite element modeling or other computational modeling could then be used to calculate stresses in the blood vessel, including shear stresses. Either assumed or patient-specific estimates of blood flow conditions could be included.

In a controlled set of parameter studies on such models, the presence of the plaque could be fully or partially removed and the resulting change in stress or other outcome parameters evaluated. In another variation of the patient-specific model, the material properties of the blood vessel could be altered to partially or fully add or remove spatial variations in plaque or other material distributions. In another variation, the geometry of the vessel could be increased or decreased or altered in some manner. Subjects could be given the same geometry properties, and responses compared against computations using their own geometric properties. In another variation, the assumed flow conditions could be altered. For example, rather than using a patient-specific flow estimate, patients could be subjected to the same “nominal” flow conditions and the difference in response between the patient-specific and “nominal” conditions could be quantified. Alternatively, different types of “nominal” flow conditions could be applied to patients and their differing responses to the different loading conditions quantified. These are merely illustrative examples of how patient-specific models could be varied in order to provide additional information for diagnosis of a disease or assessment of aging, disease, or treatment. Many other possible applications and models may be utilized as may be understood by those skilled in the art.

One or more embodiments may be conveniently implemented using a general purpose or a specialized digital computer or microprocessor programmed according to the teachings of the present disclosure, as will be apparent to those skilled in the computer art.

Appropriate software coding can readily be prepared by skilled programmers based on the teachings of the present disclosure, as will be apparent to those skilled in the software art. The embodiments may also be implemented by the preparation of application-specific integrated circuits or by interconnecting an appropriate network of component circuits, as will be readily apparent to those skilled in the art based on the present disclosure.

A computer program product is also provided including a storage medium (media) having instructions stored thereon/in which can be used to control, or cause, a computer to perform processes in accordance with preferred embodiments. The storage medium can include, but is not limited to, any type of disk including floppy disks, mini disks (MD's), optical discs, DVD, CD-ROMS, CDRW+/−, micro-drive, and magneto-optical disks, ROMs, RAMs, EPROMs, EEPROMs, DRAMs, VRAMs, flash memory devices (including flash cards, memory sticks), magnetic or optical cards, MEMS, nanosystems (including molecular memory ICs), RAID devices, remote data storage/archive/warehousing, or any type of media or device suitable for storing instructions and/or data.

Stored on one or more computer readable media, embodiments include software for controlling both the hardware of the general purpose/specialized computer or microprocessor, and for enabling the computer or microprocessor to interact with a human user or other mechanism utilizing the results. Such software may include, but is not limited to, device drivers, operating systems, and user applications. Ultimately, such computer readable media further includes software for performing methods as described above.

Those skilled in the art will appreciate that various adaptations and modifications of the just-described preferred embodiments can be configured without departing from the scope and spirit of the invention. Therefore, it is to be understood that, within the scope of the appended claims, the invention may be practiced other than as specifically described herein. The present invention is not limited to the embodiments described above herein, which may be amended or modified without departing from the scope of the present invention as set forth in the appended claims, and structural and functional equivalents thereof.

In methods that may be performed according to preferred embodiments herein and that may have been described above and/or claimed below, the operations have been described in selected typographical sequences. However, the sequences have been selected and so ordered for typographical convenience and are not intended to imply any particular order for performing the operations.

In addition, all references cited above herein, in addition to the background and summary of the invention sections, are hereby incorporated by reference into the detailed description of the preferred embodiments as disclosing alternative embodiments and components.

What is claimed is:

1. A method for determining one or more structure-function characteristics of a structure in a human or animal body from an image of the structure, comprising:
receiving an image of a structure in a body;
generating a structural model of the structure based on the image;
computing a first biomechanical quantity based on the structural model;
modifying the structural model to create a variant model;
computing a second biomechanical quantity based on the variant model;
comparing the first and second biomechanical quantities; and
storing a result of the comparing in a digital medium.

2. The method of claim 1, further comprising determining one or more structure-function characteristics based on the comparing.

3. The method of claim 1, further comprising repeating the method for altered loading conditions.

4. The method of claim 1, further comprising repeating the method at a later time period, and determining one or more effects of treatment, aging or disease, or combinations thereof, on one or more structure-function characteristics of the structure.

5. The method of claim 1, wherein the structure comprises a musculoskeletal tissue or organ or joint, or combinations thereof.

6. The method of claim 5, wherein the structure comprises bone.

7. The method of claim 1, wherein the structure comprises a cardiovascular tissue or organ, or combination thereof.

8. The method of claim 7, wherein the structure comprises a heart or blood vessel, or both.

9. The method of claim 1, wherein the variant model comprises a homogenized model, and the method further comprises assigning an average density to one or more elements of the structural model.

10. The method of claim 1, wherein the variant model comprises a reference model, and the method further comprises assigning a reference density to one or more elements of the structural model.

11. The method of claim 1, wherein the variant model comprises a sub-structure model, and the method further comprises removing a portion of structure from the model.

12. The method of claim 11, wherein the portion of structure that is removed comprises peripheral structure.

13. The method of claim 1, wherein the structural model comprises a finite element model of the structure.

14. The method of claim 1, wherein the variant model comprises a combination of two of more of a homogenized model, a sub-structure model, and a reference model.

15. The method of claim 1, wherein the variant model comprises a variation of the structural model wherein a boundary condition is modified.

16. The method of claim 15, wherein the boundary condition comprises force, pressure, bending moment, deformation, displacement, velocity, acceleration, fluid flow, temperature, energy, strain, or stress, or combinations thereof.

17. The method of claim 1, further comprising scanning the structure to create the image of the structure.

18. The method of claim 17, wherein the scanning comprises computed topography, magnetic resonance, DXA, X-ray radiograph, ultrasound, or PET scanning, or combinations thereof.

19. The method of claim 1, further comprising:
receiving a second image of the structure acquired at a different time period than when the first image was acquired;
generating a second structural model of the structure based on the second image;
computing a third biomechanical quantity based on the second structural model;
modifying the second structural model to create a second variant model; and
computing a fourth biomechanical quantity based on the second variant model, and wherein the comparing further comprises comparing the third and fourth biomechanical quantities.

20. The method of claim 19, wherein the comparing further comprises comparing the result of the comparing of the first and second biomechanical quantities with a result of the comparing of the third and fourth biomechanical quantities.

21. The method of claim 1, further comprising:
receiving a second image of a different portion of the body than said structure;
generating a second structural model based on the second image;
computing a third biomechanical quantity based on the second structural model;
modifying the second structural model to create a second variant model; and
computing a fourth biomechanical quantity based on the second variant model, and wherein the comparing further comprises comparing the third and fourth biomechanical quantifies.

22. The method of claim 21, wherein the comparing further comprises comparing a result of the comparing of the first and second biomechanical quantities with a result of the comparing of the third and fourth biomechanical quantities.

23. One or more processor readable storage devices having processor readable code embodied thereon, said processor readable code for programming one or more processors to perform a method for determining one or more structure-function characteristics of a structure in a human or animal body from an image of the structure, the method comprising:
receiving an image of a structure in a body;
generating a structural model of the structure based on the image;
computing a first biomechanical quantity based on the structural model;
modifying the structural model to create a variant model;
computing a second biomechanical quantity based on the variant model;
comparing the first and second biomechanical quantities; and
storing a result of the comparing in a digital medium.

24. The one or more storage devices of claim 23, the method further comprising determining one or more structure-function characteristics based on the comparing.

25. The one or more storage devices of claim 23, the method further comprising repeating the method for altered loading conditions.

26. The one or more storage devices of claim 23, the method further comprising repeating the method at a later time period, and determining one or more effects of treatment, aging or disease, or combinations thereof, on one or more structure-function characteristics of the structure.

27. The one or more storage devices of claim 23, wherein the structure comprises a musculo-skeletal tissue or organ, or combination thereof.

28. The one or more storage devices of claim 27, wherein the structure comprises bone.

29. The one or more storage devices of claim 23, wherein the structure comprises a cardio-vascular tissue or organ, or combination thereof.

30. The one or more storage devices of claim 29, wherein the structure comprises a heart or blood vessel, or both.

31. The one or more storage devices of claim 23, wherein the variant model comprises a homogenized model, and the method further comprises assigning an average density to one or more elements of the structural model.

32. The one or more storage devices of claim 23, wherein the variant model comprises a reference model, and the method further comprises assigning a reference density to the structural model.

33. The one or more storage devices of claim 23, wherein the variant model comprises a sub-structure model, and the method further comprises removing a portion of bone from the model.

34. The one or more storage devices of claim 33, wherein the portion of bone that is removed comprises peripheral bone.

35. The one or more storage devices of claim 23, wherein the structural model comprises a finite element model of the structure.

36. The one or more storage devices of claim 23, wherein the variant model comprises a combination of two of more of a homogenized model, a sub-structure model, an axial model, a bend model, and a reference model.

37. The one or more storage devices of claim 23, wherein the variant model comprises a variation of the structural model wherein a boundary condition is modified.

38. The one or more storage devices of claim 37, wherein the boundary condition comprises force, pressure, deformation, fluid field, energy, or stress, or combinations thereof.

39. The one or more storage devices of claim 23, further comprising scanning the structure to create the image of the structure.

40. The one or more storage devices of claim 39, wherein the scanning comprises computed topography, magnetic resonance, DXA, X-ray radiograph, ultrasound, or PET scanning, or combinations thereof.

41. The one or more storage devices of claim 23, further comprising:
receiving a second image of the structure acquired at a different time period than when the first image was acquired;
generating a second structural model of the structure based on the second image;
computing a third biomechanical quantity based on the second structural model;
modifying the second structural model to create a second variant model; and
computing a fourth biomechanical quantity based on the second variant model, and wherein the comparing further comprises comparing the third and fourth biomechanical quantities.

42. The one or more storage devices of claim 41, wherein the comparing further comprises comparing the result of the comparing of the first and second biomechanical quantities with a result of the comparing of the third and fourth biomechanical quantities.

43. The one or more storage devices of claim 23, further comprising:
receiving a second image of a different portion of said structure;
generating a second structural model based on the second image;
computing a third biomechanical quantity based on the second structural model;
modifying the second structural model to create a second variant model; and
computing a fourth biomechanical quantity based on the second variant model, and wherein the comparing further comprises comparing the third and fourth biomechanical quantities.

44. The one or more storage devices of claim 43, wherein the comparing further comprises comparing a result of the comparing of the first and second biomechanical quantities with a result of the comparing of the third and fourth biomechanical quantities.

45. A method for assessing the effects of aging, disease or treatment on a structure in a human or animal body, the method comprising:
receiving an image of a structure in a body at a first time period;
generating a structural model of the structure based on the image;
computing a first biomechanical quantity based on the structural model;
modifying the structural model to create a variant model;
computing a second biomechanical quantity based on the variant model;
comparing the first and second biomechanical quantities;
determining one or more structure-function characteristics based on comparing the first and second biomechanical quantities;
receiving a second image of the structure acquired at a second time period;
generating a second structural model of the structure based on the second image;
computing a third biomechanical quantity based on the second structural model;
modifying the second structural model to create a second variant model;
computing a fourth biomechanical quantity based on the second variant model;
comparing the third and fourth biomechanical quantities; and
comparing a result of the comparing of the first and second biomechanical quantities with a result of the comparing of the third and fourth biomechanical quantities, in order to assess and/or diagnose the effects of aging, disease or treatment on the structure.
